(19) United States
(12) Patent Application Publication
(10) Pub. No.: US 2024/0058549 A1
(43) Pub. Date: Feb. 22, 2024

(54) MEDICINAL LIQUID INJECTION APPARATUS AND MEDICINAL LIQUID INJECTION PREPARATION METHOD

(71) Applicant: Yong Hyun Kim, Gyeonggi-do (KR)

(72) Inventor: Yong Hyun Kim, Gyeonggi-do (KR)

(21) Appl. No.: 17/767,562

(22) PCT Filed: Oct. 16, 2020
(86) PCT No.: PCT/KR2020/014153

§ 371 Date: Apr. 8, 2022

(30) Foreign Application Priority Data
Oct. 18, 2019 (KR) 10-2019-0130170
Jan. 23, 2020 (KR) 10-2020-0009361

(51) Int. Cl. A61M 5/36; A61M 5/168; A61M 5/165

(52) U.S. Cl. CPC A61M 5/36; A61M 5/16877; A61M 5/165

(57) ABSTRACT

A medicinal liquid injection apparatus according to an embodiment of the present disclosure includes: a medicinal liquid storage part configured to form an internal space for storing a medicinal liquid and a priming storage space extending from the internal space and having one end located at a connection point between the priming storage space and the internal space; and a medicinal liquid flow part configured to be capable of being coupled to the medicinal liquid storage part, the medicinal liquid flow part having a guide flow path for the medicinal liquid that is configured to be capable of being connected to the other end of the priming storage space in a state that the medicinal liquid flow part is coupled to the medicinal liquid storage part.

### TECHNICAL FIELD

The present disclosure relates to a medicinal liquid injection apparatus and a medicinal liquid injection preparation method.

### BACKGROUND

In order to supply a drug to a patient, a medicinal liquid injection apparatus for injecting a liquid medicinal liquid (e.g., an injection solution) to a patient is known. Using the medicinal liquid injection apparatus, the medicinal liquid in a predetermined storage space is caused to pass through a passage (e.g., the internal spaces of a tube and an injection needle) connected to the patient and flow into the body of the patient.

When the medicinal liquid is filled in the storage space, air may be introduced together with the medicinal liquid and may be moved through the passage. In addition, depending on the conditions such as a pressure in the passage and the like, a substance (e.g., dissolved oxygen or dissolved carbon dioxide) dissolved in the medicinal liquid may become air and may move along the passage.

An inside of a tube and the like of the medicinal liquid injection apparatus is initially filled with air. Before the medicinal liquid injection apparatus is connected to the patient, a priming operation of filling the tube with a saline or a medicinal liquid to remove the air existing in the tube is known.

### SUMMARY

Various embodiments of the present disclosure provide a device for preventing occurrence of injection interruption due to air bubbles inside the device when injecting a medicinal liquid into a human body.

Various embodiments of the present disclosure provide a device designed to simplify priming which is a process for removing air bubbles inside the device.

Various embodiments of the present disclosure provide a method capable of simplifying priming which is a process for removing air bubbles inside a device.

An aspect of the present disclosure provides embodiments of a medicinal liquid injection apparatus. A medicinal liquid injection apparatus according to a representative embodiment includes: a medicinal liquid storage part configured to form an internal space for storing a medicinal liquid and a priming storage space extending from the internal space and having one end located at a connection point between the priming storage space and the internal space; and a medicinal liquid flow part configured to be capable of being coupled to the medicinal liquid storage part, the medicinal liquid flow part having a guide flow path for the medicinal liquid that is configured to be capable of being connected to the other end of the priming storage space in a state that the medicinal liquid flow part is coupled to the medicinal liquid storage part. The medicinal liquid storage part includes a medicinal liquid pumping device configured to pressurize the medicinal liquid in the internal space, and a medicinal liquid injection valve disposed at the other end of the priming storage space and configured to be capable of opening and closing the priming storage space. The medicinal liquid flow part includes a coupling member configured to be capable of being coupled to the medicinal liquid injection valve, and a medicinal liquid flow line extending from the coupling member to form the guide flow path. The medicinal liquid injection apparatus is configured such that when the coupling member is coupled to the medicinal liquid injection valve, the medicinal liquid injection valve is opened by the coupling member to allow the other end of the priming storage space to be connected to the guide flow path.

Another aspect of the present disclosure provides embodiments of a medicinal liquid injection preparation method. A medicinal liquid injection preparation method according to a representative embodiment includes: storing a medicinal liquid by introducing the medicinal liquid into an internal space of a medicinal liquid pumping device through a medicinal liquid injection valve; storing a priming liquid by introducing a priming liquid toward the internal space through the medicinal liquid injection valve; connecting a flow path by coupling a medicinal liquid flow part to the medicinal liquid injection valve to connect a guide flow path of the medicinal liquid flow part to the internal space; and priming by pressurizing, by the medicinal liquid pumping device, the internal space to allow the priming liquid flow ahead of the medicinal liquid along the guide flow path.

According to the present disclosure, it is possible to simplify priming which is a process for removing air bubbles inside a medicinal liquid injection apparatus.

### BRIEF DESCRIPTION OF THE DRAWINGS

FIG. 1 is a conceptual diagram showing an entire system of a medicinal liquid injection apparatus 1 according to a first embodiment of the present disclosure.

FIG. 2 is a partial cutaway elevation view of a medicinal liquid injection apparatus 2 according to a second embodiment of the present disclosure.

FIG. 3 is a partial cutaway elevation view showing the next state according to a use preparation process of the medicinal liquid injection apparatus 2 shown in FIG. 2.

FIG. 4 is a partial cutaway elevation view showing the next state according to the use preparation process of the medicinal liquid injection apparatus 2 shown in FIG. 3.

FIG. 5 is a partial cutaway elevation view showing the next state according to the use preparation process of the medicinal liquid injection apparatus 2 shown in FIG. 4.

FIG. 6 is a partial cutaway elevation view showing the next state according to the use preparation process of the medicinal liquid injection apparatus 2 shown in FIG. 5.

FIG. 7 is a partial cutaway elevation view showing the next state according to the use preparation process of the medicinal liquid injection apparatus 2 shown in FIG. 6.

FIG. 8 is a cross-sectional view showing a medicinal liquid injection apparatus 2′ according to a modified example of the second embodiment of the present disclosure.

FIG. 9 is a perspective view of the medicinal liquid injection apparatus 2′ of FIG. 8, showing a state in which a medicinal liquid storage part 10 and a medicinal liquid flow part 20 are separated from each other.

FIG. 10 is a perspective view of the medicinal liquid injection apparatus 2′ of FIG. 8, showing a state in which the medicinal liquid storage part 10 and the medicinal liquid flow part 20 are coupled to each other.

FIG. 11 is a partial cross-sectional view showing a state of a medicinal liquid injection valve 13 in FIG. 9.

FIG. 12 is a partial cross-sectional view showing a state of the medicinal liquid injection valve 13 in FIG. 10.

### DETAILED DESCRIPTION

Embodiments of the present disclosure are illustrated for the purpose of explaining the technical idea of the present disclosure. The scope of the rights according to the present disclosure is not limited to the embodiments presented below or the detailed descriptions of such embodiments.

All technical and scientific terms used in the present disclosure have the meaning generally understood by those of ordinary skill in the art to which the present disclosure pertains, unless otherwise defined. All terms used in the present disclosure are chosen for the purpose of more clearly describing the present disclosure and are not chosen to limit the scope of rights according to the present disclosure.

As used in the present disclosure, expressions such as “comprising,” “including,” “having,” and the like are to be understood as open-ended terms having the possibility of encompassing other embodiments, unless otherwise mentioned in the phrase or sentence containing such expressions.

The singular form described in the present disclosure may include a plural meaning, unless otherwise mentioned. This applies equally to the singular form recited in the claims.

The terms “first,” “second,” etc. used herein are used to distinguish a plurality of components from one another and are not intended to limit the order or importance of the relevant components.

In the present disclosure, when it is mentioned in the present disclosure that one element is “connected” or “coupled” to another element, it is to be understood that said one element may be directly connected or coupled to another element or may be connected or coupled to another element via a new additional element.

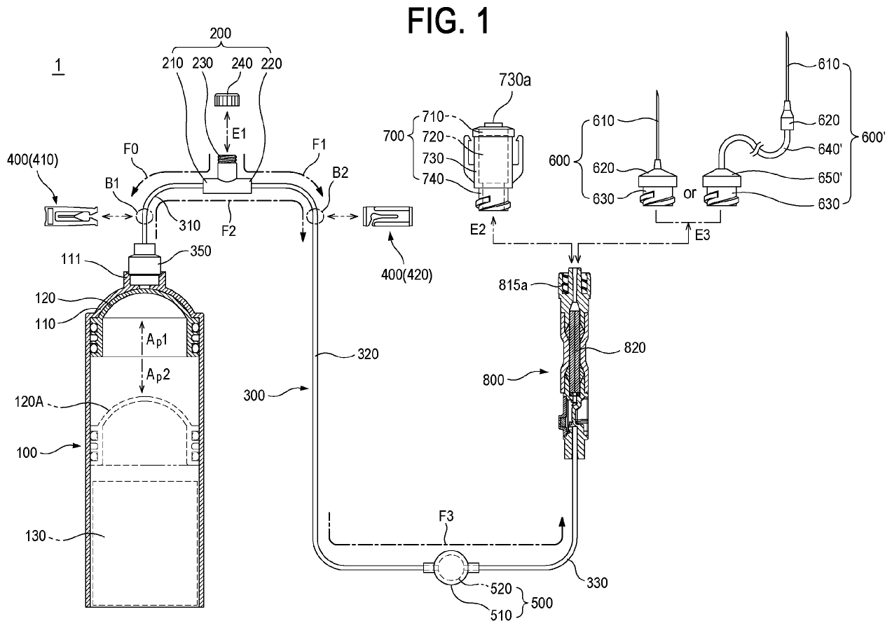

As used in the present disclosure, the terms “upstream” and “downstream” are defined based on the direction in which a medicinal liquid flows when the medicinal liquid is pumped by a medicinal liquid pumping device 100 or 11. Specifically, the direction indicated by arrows F2 and F3 in FIG. 1 is defined as downstream, and the direction opposite to the downstream is defined as upstream.

Hereinafter, descriptions are made as to embodiments of the present disclosure with reference to the accompanying drawings. In the accompanying drawings, the same or corresponding elements are denoted by the same reference numerals. In the following descriptions of the embodiments, descriptions of the same or corresponding elements may be omitted. However, even if the descriptions of elements are omitted, it is not intended that such elements are not included in a certain embodiment.

FIG. 1 is a conceptual diagram showing an entire system of a medicinal liquid injection apparatus 1 according to a first embodiment of the present disclosure. In order to inject a medicinal liquid into a patient using the medicinal liquid injection apparatus 1 according to the first embodiment of the present disclosure, a medicinal liquid injection preparation method and a medicinal liquid injection method according to a first embodiment are sequentially performed.

Referring to FIG. 1, in the medicinal liquid injection preparation method according to the first embodiment, a priming liquid is allowed to flow along an extension tube 300. The priming liquid flowing along the extension tube 300 is introduced into an external filter module 500 and a medicinal liquid delivery tube device 800 for controlling a flow rate. As an example, the medicinal liquid delivery tube device 800 may be a medicinal liquid delivery tube device in which an air filter is integrally formed.

The medicinal liquid delivery tube device 800 may include a medicinal liquid delivery tube 820 that limits a flow rate of a medicinal liquid. The medicinal liquid delivery tube 820 has a capillary flow path. The medicinal liquid delivery tube 820 may have a function of limiting the flow rate of the medicinal liquid. As an example, the medicinal liquid delivery tube 820 may include a capillary tube. As another example, the medicinal liquid delivery tube 820 may include a polymer microtube. In addition, the medicinal liquid delivery tube 820 may be composed of various shapes and materials having a capillary flow path. For example, the capillary flow path may have a diameter of about 0.04 to 0.08 mm, thereby limiting the flow rate of the medicinal liquid.

The medicinal liquid injection apparatus 1 may include an end cap 700 detachably connected to the downstream side of the medicinal liquid delivery tube device 800. The air inside the extension tube 300 may be discharged to an outside through the end cap 700.

Accordingly, the priming liquid is filled inside the extension tube 300 and the medicinal liquid delivery tube device 800. The priming liquid is a liquid different from the medicinal liquid and may be a liquid that can be injected into a patient and has a lower risk than the medicinal liquid when exposed to air. For example, the priming liquid may be a saline.

The end cap 700 is configured such that the air and the priming liquid passing through the medicinal liquid delivery tube device 800 are introduced therein. The end cap 700 may be configured to discharge the air to the outside but prevent the priming liquid from being discharged to the outside.

The end cap 700 includes a vent filter 710 that blocks the priming liquid but passes the gas. The vent filter 710 includes a hydrophobic filter. The end cap 700 may include a sponge 720 disposed on the upstream side of the vent filter 710. The end cap 700 includes an end cap casing 730 that accommodates the vent filter 710 therein. The end cap casing 730 accommodates the sponge 720 therein. The end cap casing 730 forms a vent hole 730a through which the gas passes. The end cap 700 includes an end cap coupling portion 740 configured to be capable of being coupled to a downstream connection portion 815a of the medicinal liquid delivery tube device 800. Arrow E2 in FIG. 1 indicates the coupling and separation directions of the end cap coupling portion 740 with respect to the downstream connection portion 815a.

When the priming liquid is filled in the end cap 700, the end cap 700 is separated from the medicinal liquid delivery tube device 800. The medicinal liquid delivery tube device 800 may be connected to a patient connection unit 600 or 600′.

Each of the patient connection units 600 and 600′ may include an injection needle 610, a catheter, or the like. Each of the patient connection units 600 and 600′ includes a component to be inserted into the body of the patient, such as the injection needle 610 or the like.

Each of the patient connection units 600 and 600′ may include an “insertion part” including a component to be inserted into the patient's body, such as the injection needle 610 or the like, and a ‘remaining part.’ The insertion part and the remaining part may be detachably coupled to each other. In this case, in a state in which the insertion part is connected to the patient and separated from the remaining part, the user may couple the remaining part to the medicinal liquid delivery tube device 800 and then may connect the insertion part and the remaining part to each other. In this case, the liquid passing through the medicinal liquid delivery tube device 800 may sequentially pass through the remaining part and the insertion part and may flow into the body of the patient.

Each of the patient connection units 600 and 600′ includes an injection support 620 that supports the injection needle 610. Each of the patient connection units 600 and 600′ includes a unit coupling portion 630 configured to be capable of being coupled to the downstream connection portion 815a of the medicinal liquid delivery tube device 800. Arrow E3 in FIG. 1 indicates the coupling and separation directions of the unit coupling portion 630 with respect to the downstream connection portion 815a.

As an example, the patient connection unit 600 may be configured by sequentially connecting the injection needle 610, the injection support 620, and the unit coupling portion 630.

As another example, the patient connection unit 600′ further includes a patient connection tube fixing portion 650′ connected to the downstream side of the unit coupling portion 630. The patient connection unit 600′ further includes a patient connection tube 640′ for connecting the patient connection tube fixing portion 650′ and the injection support 620. The patient connection tube 640′ may be formed of a flexible material. The patient connection unit 600′ may be configured by sequentially connecting the injection needle 610, the injection support 620, a patient connection tube 640′, a patient connection tube fixing portion 650′, and the unit coupling portion 630.

In the medicinal liquid injection method, the medicinal liquid is introduced into the body of the patient by the pumping in a medicinal liquid pumping device 100. The medicinal liquid is a liquid containing a therapeutic substance. The priming liquid may be first introduced into the body of the patient, and the medicinal liquid flowing after the priming liquid may be introduced into the body of the patient.

The medicinal liquid pumping device 100 includes a chamber 110 configured to be capable of accommodating the medicinal liquid. The chamber 110 forms an internal space together with a pressurization unit 120. The medicinal liquid may be stored in the internal space. In another embodiment, a saline solution or the like may be temporarily stored in the internal space. The chamber 110 has a discharge port portion 111 through which the liquid inside the chamber 110 is discharged.

The medicinal liquid pumping device 100 is configured to pump the medicinal liquid. The medicinal liquid pumping device 100 includes the pressurization unit 120 for pressurizing the liquid inside the chamber 110. The pressurization unit 120 may pressurize the liquid inside the chamber 110 by moving in a predetermined pressurization direction Ap1. When the liquid is being filled into the chamber 110, the pressurization unit 120 moves in a direction Ap2 opposite to the pressurization direction Ap1. In FIG. 1, reference numeral 120A indicates the position of the pressurization unit 120 moved in the opposite direction Ap2.

The medicinal liquid pumping device 100 may include a pressurization operation part 130 for providing power to move the pressurization unit 120 in the pressurization direction Ap1. For example, the pressurization operation part 130 may be configured to pressurize the liquid in the chamber 110 using volume expansion caused by gas activation. As another example, the pressurization operation part 130 may provide a portion which can be held by the user, so that the pressurization unit 120 can be moved in the pressurization direction Ap1 by the user's force.

Although not shown, as another example, the pressurization unit 120 may be configured to pressurize the liquid using the elastic force of an elastic body such as a balloon or the like. In this case, the pressurization unit 120 may be configured to pressurize the liquid in the balloon.

A medicinal liquid injection valve 200 is configured to be capable of filling the liquid into the chamber 110. The liquid may be introduced into the extension tube 300 or the chamber 110 from an outside through the medicinal liquid injection valve 200. The medicinal liquid injection valve 200 is connected to the extension tube 300. In another embodiment not shown, the medicinal liquid injection valve 200 may be connected to the chamber 110.

The medicinal liquid injection valve 200 includes a first extension portion 210 connected to a downstream end of a first connection portion 310 of the extension tube 300, and a second extension portion 220 connected to an upstream end of the second connection portion 320 of the extension tube 300. The medicinal liquid injection valve 200 includes an inlet portion 230 configured to allow a liquid to be introduced from the outside, and an inlet port opening/closing portion 240 configured to be detachably coupled to the inlet portion 230. Arrow E1 of FIG. 1 indicates the coupling and separation directions of the inlet port opening/closing portion 240 with respect to the inlet portion 230.

The extension tube 300 is configured to guide the flow of the priming liquid. The extension tube 300 may guide the movement of the medicinal liquid from the medicinal liquid pumping device 100 to the medicinal liquid delivery tube device 800.

The extension tube 300 is configured such that the medicinal liquid flowing out from the medicinal liquid pumping device 100 by the pressurization in the medicinal liquid pumping device 100 flows through the extension tube 300. An upstream end of the extension tube 300 is connected to the medicinal liquid pumping device 100. The extension tube 300 includes an upstream connection portion 350 connected to the medicinal liquid injection discharge port portion 111 of the medicinal liquid pumping device 100.

A downstream end of the extension tube 300 is connected to the medicinal liquid delivery tube device 800. The liquid passing through the extension tube 300 is introduced into the medicinal liquid delivery tube device 800 through the inlet port of the medicinal liquid delivery tube device 800.

The extension tube 300 includes the first connection portion 310 for connecting the upstream connection portion 350 and the first extension portion 210 of the medicinal liquid injection valve 200. The extension tube 300 includes a second connection portion 320 for connecting the second extension portion 220 of the medicinal liquid injection valve 200 and the external filter module 500. The extension tube 300 includes a third connection portion 330 for connecting the external filter module 500 and the downstream end of the extension tube 300.

The medicinal liquid injection apparatus 1 may include at least one connection tube opening/closing module 400. The connection tube opening/closing module 400 may press the outer surface of the extension tube 300 to block the flow of liquid at one point on the extension tube 300. The at least one connection tube opening/closing module 400 may include a first opening/closing module 410 that can open or close the first connection portion 310 at one point B1 and a second opening/closing module 420 that can open or close the second connection portion 320 at one point B2. For example, the connection tube opening/closing module 400 may be configured in a clamp shape.

The medicinal liquid injection apparatus 1 may include the external filter module 500 disposed on the extension tube 300. The external filter module 500 may include a filter casing 510 connected to the extension tube 300 and a filter 520 disposed in the filter casing 510. As the filter 520 of the external filter module 500, a particle filter for filtering impurities, an air filter for filtering air bubbles, or a combination thereof may be used. An air vent configured to discharge the air filtered in the air filter to the outside may be formed in the external filter module 500.

A medicinal liquid injection preparation method and a medicinal liquid injection method according to a first embodiment will be described below. In the medicinal liquid injection preparation method according to the first embodiment, the inlet port opening/closing portion 240 is separated from the inlet portion 230, the first connection portion 310 is blocked with the first connection tube opening/closing module 410 (see B1), and the rest of the extension tube 300 except for the first connection portion 310 is opened. As indicated by arrows F1 and F3, the priming liquid such as a saline or the like flows sequentially through the inlet portion 230, the second extension portion 220, the second connection portion 320, the third connection portion 330, and the medicinal liquid delivery tube device 800, whereby an inside of the extension tube 300 and an inside of the medicinal liquid delivery tube device 800 are filled with the priming liquid.

After performing the medicinal liquid injection preparation method according to the first embodiment, the medicinal liquid injection method according to the first embodiment is performed. In the medicinal liquid injection method according to the first embodiment, the inlet port opening/closing portion 240 is separated from the inlet portion 230, the second connection portion 320 is blocked with the second connection tube opening/closing module 420 (see B2), and the first connection tube opening/closing module 410 is separated from the first connection portion 310 to open the first connection portion 310. As indicated by arrow F0, as the medicinal liquid flows into the chamber 110 through the medicinal liquid injection valve 200 and the first connection portion 310, the pressurization unit 120 moves in the direction Ap2. Thereafter, the inlet port opening/closing portion 240 is coupled to the inlet portion 230, and the second connection tube opening/closing module 420 is separated from the second connection portion 320 to open the extension tube 300. As indicated by arrows F2 and F3, the pressurization unit 120 is then moved in the pressurization direction Ap1, so that the medicinal liquid can sequentially pass through the extension tube 300 and the medicinal liquid delivery tube device 800.

The medicinal liquid injection apparatus 1 according to the first embodiment described above has a problem that in order to perform the priming, the priming liquid (e.g., a saline) needs to be injected by pressurizing a syringe for a long time in a state in which the syringe is connected to the inlet portion 230. In addition, the medicinal liquid injection apparatus 1 according to the first embodiment has a problem that the air existing inside the first connection portion 310 connecting the upstream connection portion 350 and the first extension portion 210 cannot be removed in the medicinal liquid injection preparation method according to the first embodiment.

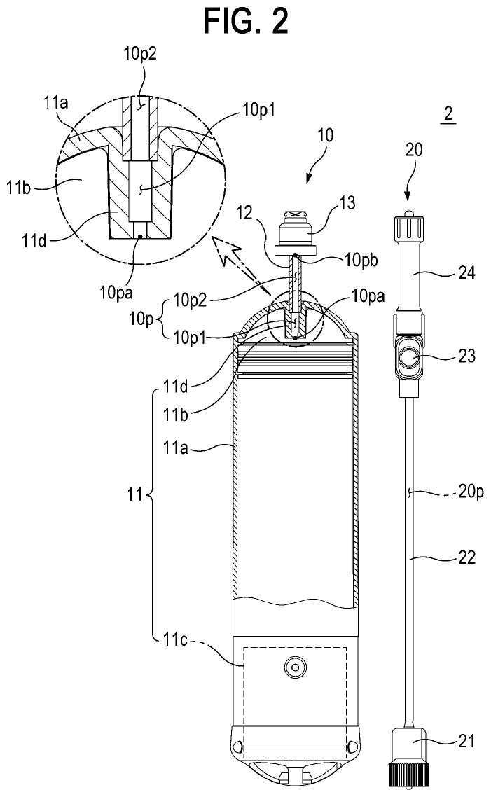

In contrast, a medicinal liquid injection apparatus 2 according to a second embodiment, which will be described below with reference to FIGS. 2 to 12, makes it convenient to inject the priming liquid and eliminate or reduce a phenomenon of the air remaining inside a tube due to priming. Hereinafter, the medicinal liquid injection apparatus 2 and the medicinal liquid injection preparation method according to the second embodiment will be described with reference to FIGS. 2 to 12, by focusing on the differences from the first embodiment described above.

FIG. 2 is a view showing the medicinal liquid injection apparatus 2 according to the second embodiment of the present disclosure and is an elevation view in which a part of the medicinal liquid injection apparatus 2 is cut away. Referring to FIG. 2, the medicinal liquid injection apparatus 2 is provided in a state in which the medicinal liquid storage part 10 and a medicinal liquid flow part 20 are separated from each other. The medicinal liquid storage part 10 and the medicinal liquid flow part 20 are capable of being connected to each other. The medicinal liquid storage part 10 and the medicinal liquid flow part 20 may be configured such that, once connected, they cannot be separated by a general method of use.

In the medicinal liquid injection apparatus 2, the medicinal liquid storage part 10 and the medicinal liquid flow part 20 may be separated from each other at the boundary between the medicinal liquid injection valve 13 and a coupling member 21. The medicinal liquid storage part 10 may include a medicinal liquid pumping device 11, a medicinal liquid storage line 12, and a medicinal liquid injection valve 13. The medicinal liquid flow part 20 may include the coupling member 21 configured to be capable of being coupled to the medicinal liquid injection valve 13, a medicinal liquid flow line 22, an air filter 23, and a medicinal liquid delivery tube device 24. The medicinal liquid injection apparatus 2 may include the end cap 700 detachably coupled to a downstream end of the medicinal liquid flow part 20. In the present embodiment, the end cap 700 may be detachably coupled to a downstream end of the medicinal liquid delivery tube device 24.

In the second embodiment, by operating the medicinal liquid pumping device 11 once, it is possible to perform both a priming step and a medicinal liquid injection step of injecting a medicinal liquid into a patient, which will be described later. The priming step constitutes a part of a medicinal liquid injection preparation method according to a second embodiment to be described later, and the medicinal liquid injection step is performed after performing the medicinal liquid injection preparation method according to the second embodiment. In a state in which the end cap 700 is coupled to the downstream end of the medicinal liquid flow part 20, the priming step may be performed. The priming step is performed in a state in which the patient and the medicinal liquid injection apparatus are separated from each other.

After the priming step, in order to inject the medicinal liquid into the patient, the end cap 700 may be separated from the downstream end of the medicinal liquid flow part 20, and the patient connection unit 600 or 600′ may be connected to the downstream end of the medicinal liquid flow part 20. The medicinal liquid injection step may be performed in a state in which the medicinal liquid injection apparatus is connected to the patient.

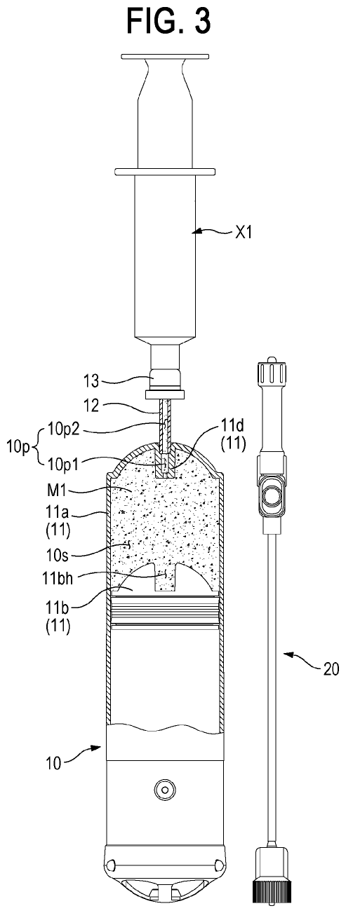

Referring to FIGS. 2 and 3, the medicinal liquid storage part 10 is configured to form an internal space 10s for storing the medicinal liquid. The internal space 10s may be formed inside the chamber 110 of the medicinal liquid storage part 10. The internal space 10s may be formed by the chamber 110 and the pressurization unit 120 of the medicinal liquid storage part 10. The medicinal liquid may be filled in the internal space 10s.

A priming storage space 10p extending from the internal space 10s is formed in the medicinal liquid storage part 10. One end 10pa of the priming storage space 10p may be located at a connection point between the priming storage space 10p and the internal space 10s. The other end 10pb of the priming storage space 10p may be located at a connection point between the priming storage space 10p and the medicinal liquid injection valve 13. The priming liquid may be filled in the priming storage space 10p. The priming liquid injected into the medicinal liquid storage part in a priming liquid storage step to be described later may be stored in the priming storage space. In the priming step to be described later, the priming liquid in the priming storage space may move to the medicinal liquid flow line 22 of the medicinal liquid flow part 20 to prime the entire medicinal liquid flow part 20.

The priming storage space 10p may be a single flow path extending from the one end 10pa to the other end 10pb. Accordingly, the medicinal liquid can be introduced into or discharged from the internal space 10s through the priming storage space 10p, and the priming storage space 10p can be used as a space in which the priming liquid is stored.

The priming storage space 10p may be formed to be longer in the direction of movement of the medicinal liquid than the width in a direction perpendicular to the direction of movement of the medicinal liquid (i.e., up-and-down direction) in the priming storage space 10p. Accordingly, when the medicinal liquid is pressurized in the priming step to be described later, it may be difficult for a part of the medicinal liquid to move to the downstream side ahead of the priming liquid in the priming storage space 10p.

The priming storage space 10p may include a partial space 10p1 in the medicinal liquid pumping device 11 and/or a space 10p2 in the medicinal liquid storage line 12. The priming storage space 10p may include the inner storage space 10p1 extending upstream inside the medicinal liquid pumping device 11 and including the one end 10pa of the priming storage space 10p. The priming storage space 10p may include the outer storage space 10p2 extending downstream outside the medicinal liquid pumping device 11 and including the other end 10pb of the priming storage space 10p. In the present embodiment, an upstream end of the outer storage space 10p2 is connected to a downstream end of the inner storage space 10p1 to form the priming storage space 10p.

The medicinal liquid injection valve 13 is disposed at the other end 10pb of the priming storage space 10p. The medicinal liquid injection valve 13 may be coupled to a downstream end of the medicinal liquid storage line 12. The medicinal liquid injection valve 13 is configured to be capable of opening and closing the priming storage space 10p. The medicinal liquid injection valve 13 may be configured to open the flow path when the tip (i.e., a downstream end) thereof is pressurized in the upstream direction, and close the flow path when the pressurization is released. The medicinal liquid injection valve 13 may be configured to open the flow path when pressurized by a syringe X1 or X2 to be described later, and close the flow path when the pressurization from the syringe X1 or X2 is released. The medicinal liquid injection valve 13 may be configured to be capable of being coupled to the coupling member 21 of the medicinal liquid flow part 20. The medicinal liquid injection valve 13 may be configured to connect the priming storage space 10p and a guide flow path 20p by opening the flow path when the medicinal liquid injection valve 13 is coupled to the coupling member 21.

The medicinal liquid storage line 12 is coupled to the medicinal liquid pumping device 11. An upstream end of the medicinal liquid storage line 12 may be coupled to the medicinal liquid pumping device 11. The downstream end of the medicinal liquid storage line 12 may be coupled to the medicinal liquid injection valve 13. The outer storage space 10p2 may be formed in the medicinal liquid storage line 12. For example, the medicinal liquid storage line 12 may be a flexible tube.

The medicinal liquid pumping device 11 is configured to be capable of accommodating the medicinal liquid in the internal space 10s. The medicinal liquid pumping device 11 is configured to be capable of pressurizing the medicinal liquid in the internal space 10s.

The medicinal liquid pumping device 11 includes a chamber 11a configured to be capable of accommodating the medicinal liquid therein. The chamber 110 forms the internal space 10s together with the pressurization unit 120. The medicinal liquid may be stored in the internal space 10s.

The medicinal liquid pumping device 11 includes a pressurization unit 11b that forms the internal space 10s together with the chamber. The pressurization unit 11b is configured to be capable of pressurizing the medicinal liquid in the internal space 10s. The pressurization unit 11b may pressurize the medicinal liquid in the internal space 10s by moving in a predetermined pressurization direction. When the medicinal liquid is being filled into the chamber 11a, the pressurization unit 120 may move in a direction opposite to the pressurization direction.

The medicinal liquid pumping device 11 may include a pressurization operation part 11c configured to provide power to move the pressurization unit 11b in the pressurization direction. The description of the pressurization operation part 11c is the same as the description of that in the above-described first embodiment.

The medicinal liquid pumping device 11 may include an inner storage part 11d that forms the inner storage space 10p1. The inner storage part 11d may extend in a direction facing the pressurization unit 11b from the inner surface of the chamber 11a. The inner storage part 11d may extend in an upstream direction from the inner surface of the chamber 11a. The internal space 10s and the inner storage space 10p1 are connected to each other through an opening at an upstream end of the inner storage part 11d. The inner storage part 11d may be integrally formed with the chamber 11a.

A storage corresponding groove 11bh may be formed in the pressurization unit 11b to engage the inner storage part 11d. A portion of the pressurization surface (the surface facing the internal space 10s) of the pressurization unit 11b may be depressed to form the storage corresponding groove 11bh. The storage corresponding groove 11bh may constitute a part of the internal space 10s. Accordingly, the internal space 10s of the medicinal liquid pumping device 11 can be efficiently formed.

The medicinal liquid flow part 20 is configured to be capable of being coupled to the medicinal liquid storage part 10. The medicinal liquid flow part 20 has the guide flow path 20p for the medicinal liquid configured to be capable of being connected to the other end 10pb of the priming storage space 10p in a state in which it is coupled to the medicinal liquid storage part 10. In a state in which the medicinal liquid flow part 20 and the medicinal liquid storage part 10 are coupled to each other, an upstream end of the guide flow path 20p and a downstream end of the priming storage space 10p are connected to each other with the medicinal liquid injection valve 13 interposed therebetween. In the medicinal liquid injection step, a downstream end of the guide flow path 20p may be connected to the patient connection unit 600 or 600′ by the user.

The coupling member 21 may be configured to be capable of being coupled to the medicinal liquid injection valve 13. The coupling member 21 is disposed at an upstream end of the medicinal liquid flow line 22. An upstream end portion of the guide flow path 20p may be formed in the coupling member 21.

The medicinal liquid flow line 22 extends from the coupling member 21. The upstream end of the medicinal liquid flow line 22 may be coupled to the coupling member 21. A downstream end of the medicinal liquid flow line 22 may be coupled to the air filter 23 or the medicinal liquid delivery tube device 24. The guide flow path 20p is formed in the medicinal liquid flow line 22. That is, a part or the entirety of the guide flow path 20p may be formed in the medicinal liquid flow line 22. For example, the medicinal liquid flow line 22 may be a flexible tube.

The medicinal liquid injection apparatus 2 is configured such that, when the coupling member 21 is coupled to the medicinal liquid injection valve 13, the medicinal liquid injection valve 13 is opened by the coupling member 21 to connect the other end 10pb of the priming storage space 10p and the guide flow path 20p.

The air filter 23 may filter air bubbles from the liquid flowing through the guide flow path 20p. The air filter 23 may be combined with a particle filter that filters out impurities. An air vent configured to discharge the air filtered by the air filter 23 to the outside may be formed in the air filter 23.

The medicinal liquid delivery tube device 24 is configured to adjust the flow rate of the liquid flowing through the guide flow path 20p. In the present embodiment, the medicinal liquid delivery tube device 24 is formed integrally with the air filter 23. However, in an embodiment not shown, the medicinal liquid delivery tube device 24 and the air filter 23 may be configured separately from each other, in which case the air filter 23 may be disposed in the middle of the medicinal liquid flow line 22.

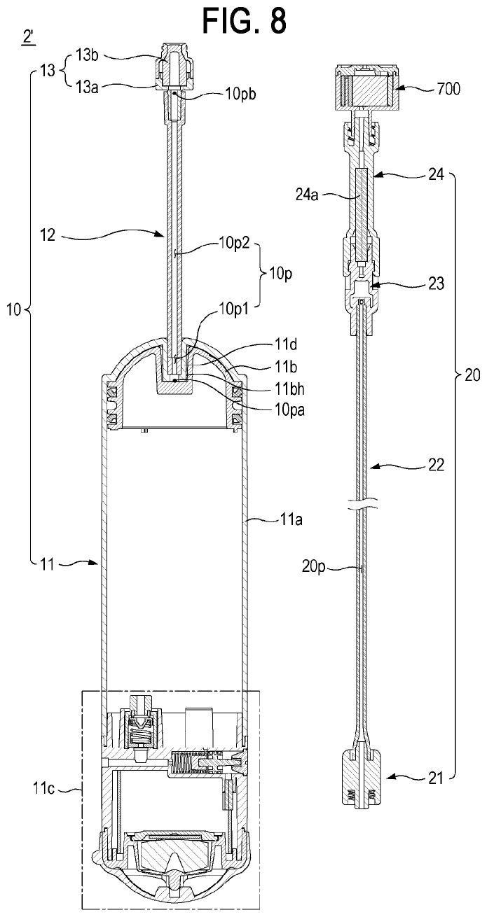

The medicinal liquid delivery tube device 24 may include a medicinal liquid delivery tube 24a for limiting the flow rate of the medicinal liquid (see FIG. 8). The medicinal liquid delivery tube 24a has a capillary flow path constituting a part of the guide flow path 20p. The description of the medicinal liquid delivery tube 24a is the same as the description of the above-described medicinal liquid delivery tube 820.

The end cap 700 may be configured to be capable of being coupled to a front end (i.e., the downstream end) of the medicinal liquid flow part 20. The end cap 700 may be disposed at the downstream end of the guide flow path 20p. In the present embodiment, the end cap 700 is configured to be capable of being coupled to the downstream end of the medicinal liquid delivery tube device 24.

In the priming step to be described later, the air inside the guide flow path 20p may be discharged to the outside through the end cap 700. Accordingly, the priming liquid may be filled in the guide flow path 20p.

The end cap 700 is configured such that the air passing through the guide flow path 20p and the priming liquid can be introduced into the end cap 700. The end cap 700 may be configured to discharge the air to the outside but prevent the priming liquid from flowing to the outside. The description of an example of a specific configuration of the end cap 700 is the same as that of the end cap according to the first embodiment described above.

When the priming liquid is filled in the end cap 700, the end cap 700 may be separated from the medicinal liquid flow part 20, and the medicinal liquid flow part 20 may be connected to the patient connection unit 600 or 600′.

FIGS. 2 to 7 are elevation views in which a part of the medicinal liquid injection apparatus 2 according to the second embodiment of the present disclosure is cut away, and are views sequentially showing a use preparation process for the medicinal liquid injection apparatus 2.

Hereinafter, a medicinal liquid injection preparation method according to a second embodiment of the present disclosure will be described with reference to FIGS. 2 to 7. The medicinal liquid injection preparation method may be performed in the order of FIGS. 2 to 7. The medicinal liquid injection preparation method includes a medicinal liquid storage step, and a priming liquid storage step performed after the medicinal liquid storage step. The medicinal liquid injection preparation method further includes a flow path connection step and a priming step that are performed after the priming liquid storage step.

Referring to FIG. 2, the medicinal liquid injection preparation method may include a step of preparing the medicinal liquid injection apparatus 2 in a state in which the medicinal liquid storage part 10 and the medicinal liquid flow part 20 are separated from each other. In this case, the priming storage space 10p is filled with air.

Referring to FIG. 3, the medicinal liquid injection preparation method includes the medicinal liquid storage step of subsequently filling the medicinal liquid storage part 10 with the medicinal liquid M1. In the medicinal liquid storage step, the medicinal liquid M1 is introduced into the internal space 10s of the medicinal liquid pumping device 11 through the medicinal liquid injection valve 13. In the medicinal liquid storage step, the syringe X1 is operated to fill the medicinal liquid M1 of the syringe X1 into the medicinal liquid pumping device 11 in a state in which the syringe X1 filled with the medicinal liquid M1 is coupled to the medicinal liquid injection valve 13 (e.g., a state in which the syringe and the medicinal liquid injection valve are pressed against each other by the user's force, or a state in which the syringe and the medicinal liquid injection valve are structurally coupled to each other). In a state in which the syringe X1 is coupled to the medicinal liquid injection valve 13, the medicinal liquid injection valve 13 is opened. In the medicinal liquid storage step, the medicinal liquid M1 may be introduced into the internal space 10s through the priming storage space 10p that connects the medicinal liquid injection valve 13 and the internal space 10s. For example, the medicinal liquid M1 may be 5FU.

Thereafter, the syringe X1 is separated from the medicinal liquid storage part 10. In the state in which the syringe X1 is separated from the medicinal liquid injection valve 13, the medicinal liquid injection valve 13 is closed.

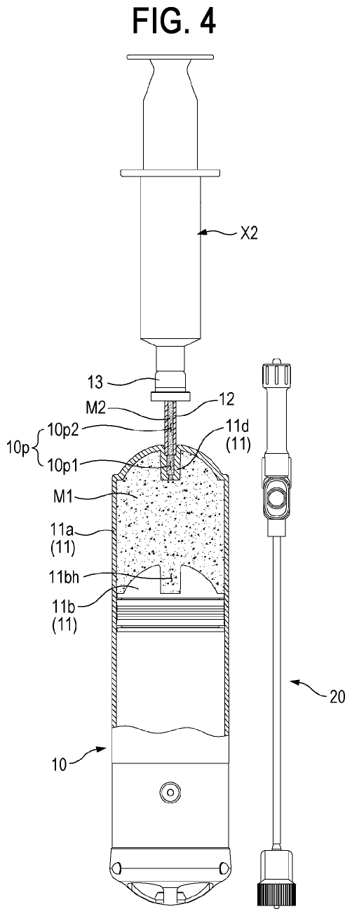

Referring to FIG. 4, the medicinal liquid injection preparation method includes the priming liquid storage step of additionally filling the medicinal liquid storage part 10 with the priming liquid M2. In the priming liquid storage step, the priming liquid M2 is introduced toward the internal space 10s through the medicinal liquid injection valve 13. In the priming liquid storage step, the priming storage space 10p may be filled with the priming liquid M2. In the priming liquid storage step, the syringe X2 is operated to fill the priming liquid M2 of the syringe X2 into the priming storage space 10p in a state in which the syringe X2 filled with the priming liquid M2 is coupled to the medicinal liquid injection valve 13 (e.g., a state in which the syringe and the medicinal liquid injection valve are pressed against each other by the user's force, or a state in which the syringe and the medicinal liquid injection valve are structurally coupled to each other). In a state in which the syringe X2 is coupled to the medicinal liquid injection valve 13, the medicinal liquid injection valve 13 is opened. For example, the priming liquid M2 may be a saline.

Thereafter, the syringe X2 is separated from the medicinal liquid storage part 10. In a state in which the syringe X2 is separated from the medicinal liquid injection valve 13, the medicinal liquid injection valve 13 is closed.

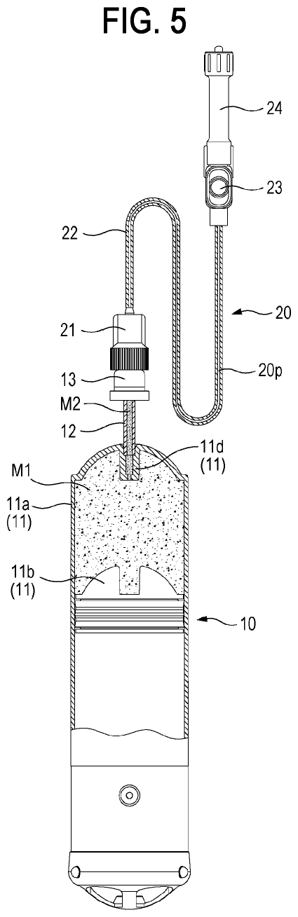

Referring to FIG. 5, the medicinal liquid injection preparation method includes the flow path connection step of subsequently connecting the medicinal liquid storage part 10 and the medicinal liquid flow part 20 to each other. In the flow path connection step, the medicinal liquid flow part 20 is coupled to the medicinal liquid injection valve 13 to connect the guide flow path 20p of the medicinal liquid flow part 20 and the internal space 10s. In this case, the medicinal liquid injection valve 13 and the coupling member 21 may be coupled to each other (see FIG. 12). In a state in which the coupling member 21 is coupled to the medicinal liquid injection valve 13, the medicinal liquid injection valve 13 is opened. In this case, the end cap 700 may be coupled to the downstream end of the medicinal liquid flow part 20, and the medicinal liquid flow part 20 may be provided in a state in which it is coupled with the end cap 700 from the beginning (see FIG. 8).

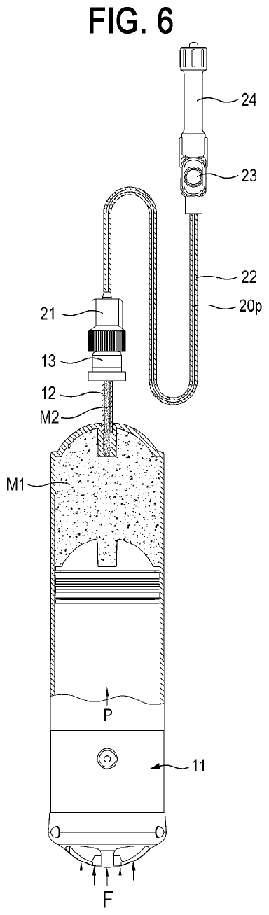

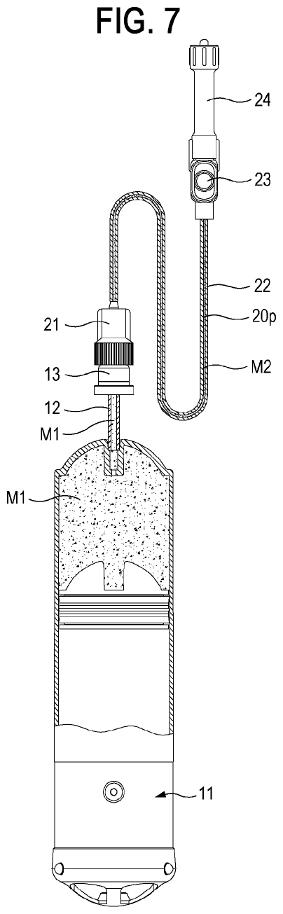

Referring to FIGS. 6 and 7, the medicinal liquid injection preparation method includes the priming step of subsequently pressurizing the medicinal liquid to allow the priming liquid M2 to flow along the guide flow path 20p. In the priming step, the medicinal liquid pumping device 11 pressurizes the internal space 10s, so that the priming liquid M2 flows ahead of the medicinal liquid M1 along the guide flow path 20p.

Referring to FIG. 6, the priming step includes a step of operating the medicinal liquid pumping device 11. The medicinal liquid pumping device 11 pressurizes the medicinal liquid M1 in the internal space 10s. The operating mechanism of the medicinal liquid pumping device of the medicinal liquid injection apparatus 1 according to the first embodiment described above may be applied. In the present embodiment, the medicinal liquid pumping device 11 generates a gas (e.g., carbon dioxide) therein to pressurize the medicinal liquid M1 by the pressure of the generated gas (see arrow P). For example, the user may press a predetermined portion of the medicinal liquid pumping device (see arrow F) to generate a gas to start the operation of the pressurization operation part 130.

Referring to FIG. 7, in the priming step, when the medicinal liquid pumping device 11 pressurizes the medicinal liquid M1, the priming liquid M2 is first filled along the guide flow path 20p. When the priming liquid M2 is filled up to the end cap 700 along the guide flow path 20p, the priming process is completed, and the preparation for injecting the medicinal liquid is completed. In a state in which the priming liquid M2 has reached the end cap 700, the medicinal liquid M1 may be introduced to the upstream side in the medicinal liquid flow part 20 or may not be introduced into the medicinal liquid flow part 20.

After the medicinal liquid injection preparation method described above is performed, the end cap 700 is separated from the medicinal liquid flow part 20, and the patient connection unit 600 or 600′ is connected to the medicinal liquid flow part 20 to perform the medicinal liquid injection step. In the medicinal liquid injection step, first, the priming liquid may be introduced into the patient's body, and the medicinal liquid flowing after the priming liquid may be introduced into the patient's body.

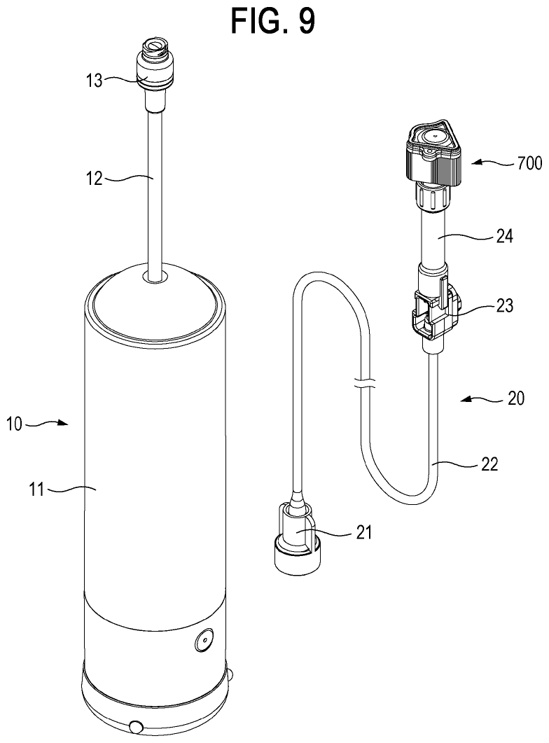

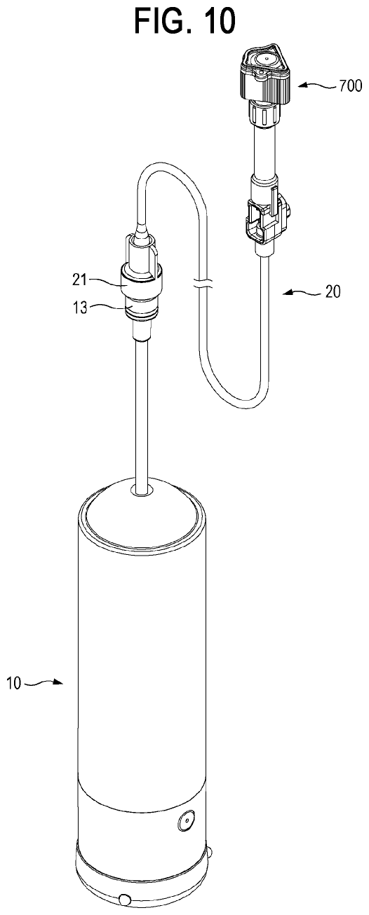

FIG. 8 is a cross-sectional view showing a medicinal liquid injection apparatus 2′ according to a modification of the second embodiment. FIGS. 9 and 10 are perspective views of the medicinal liquid injection apparatus 2′ shown in FIG. 8. FIG. 9 shows a state in which the medicinal liquid storage part 10 is separated from the medicinal liquid flow part 20, and FIG. 10 shows a state in which the medicinal liquid storage part 10 is coupled to the medicinal liquid flow part 20.

In the modification of the medicinal liquid injection apparatus 2′ according to the second embodiment shown in FIGS. 8 to 10, the medicinal liquid storage line 12 is longer than that of the embodiment shown in FIG. 2. The longer the medicinal liquid storage line 12, the larger the priming storage space 10p.

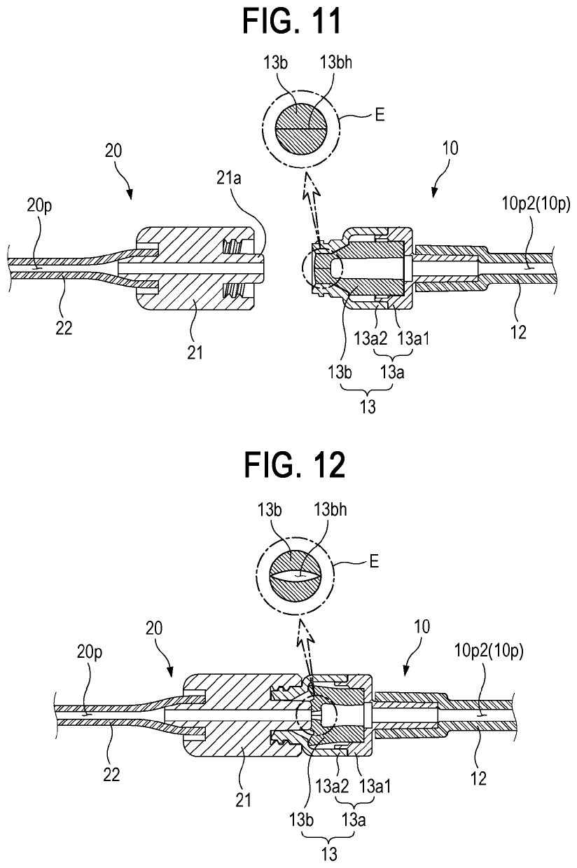

FIG. 11 is a cross-sectional view showing the state of the medicinal liquid injection valve 13 shown in FIG. 9. FIG. 12 is a cross-sectional view showing the state of the medicinal liquid injection valve 13 shown in FIG. 10. Part E in FIGS. 11 and 12 is an elevation view in which a valve part 13b is viewed in a direction in which a protrusion 21a presses the valve part 13b.

Referring to FIGS. 11 and 12, the medicinal liquid injection valve 13 may include a valve casing 13a and the valve part 13b disposed inside the valve casing 13a. The valve casing 13a may form a space for accommodating the valve part 13b therein and may have an opening formed at a front end thereof. A front end of the valve part 13b may be exposed through the opening of the valve casing 13a. The valve casing 13a may include a first part 13a1 and a second part 13a2 assembled with each other. The first part 13a1 may constitute a downstream part forming the opening, and the second part 13a2 may constitute an upstream part coupled to the medicinal liquid storage line 12.

The valve part 13b may be configured to be opened in a pressed state of the front end thereof (i.e., a pressed state of the downstream side thereof) and may be configured to be closed in a released state of the pressing. The valve part 13b may be formed of a flexible material such as rubber or the like. A hole (e.g., a slit) 13bh that can be opened in the pressed state is formed in the valve part 13b. As a hole 13bh is closed, the downstream end of the priming storage space 10p is blocked (see FIG. 11). As the hole 13bh is opened, the priming storage space 10p and the guide flow path 20p are connected to each other through the hole 13bh (see FIG. 12).

When the protrusion 21a of the coupling member 21 presses the valve part 13b in a state in which the coupling member 21 and the medicinal liquid injection valve 13 are coupled to each other, the valve part 13b is elastically deformed and the hole 13bh is opened to open the flow path. When the object pressing the valve part 13b disappears, the valve part 13b is elastically restored and the hole 13bh is closed to close the flow path.

The protrusion 21a of the coupling member 21 may be formed to protrude in an upstream direction. The protrusion 21a may be inserted into the medicinal liquid injection valve 13 through the opening of the valve casing 13a. The front end (i.e., a downstream end) of the valve casing 13a may cover the outer peripheral surface of the protrusion 21a. The thread formed on the outer peripheral surface of the front end of the valve casing 13a may be engaged with the thread formed on the inner peripheral surface of an upstream end of the coupling member 21.

The technical idea of the present disclosure has been described heretofore with reference to some embodiments and examples shown in the accompanying drawings. However, it is to be understood that various substitutions, modifications and alterations may be made without departing from the technical idea and scope of the present disclosure that can be understood by those of ordinary skill in the technical field to which the present disclosure pertains. Further, it is to be understood that such substitutions, modifications and alterations fall within the appended claims.

What is claimed is:

1. A medicinal liquid injection apparatus, comprising:
a medicinal liquid storage part configured to form an internal space for storing a medicinal liquid and a priming storage space extending from the internal space and having one end located at a connection point between the priming storage space and the internal space; and
a medicinal liquid flow part configured to be capable of being coupled to the medicinal liquid storage part, the medicinal liquid flow part having a guide flow path for the medicinal liquid that is configured to be capable of being connected to the other end of the priming storage space in a state that the medicinal liquid flow part is coupled to the medicinal liquid storage part,
wherein the medicinal liquid storage part includes:
a medicinal liquid pumping device configured to pressurize the medicinal liquid in the internal space; and
a medicinal liquid injection valve disposed at the other end of the priming storage space and configured to be capable of opening and closing the priming storage space,
wherein the medicinal liquid flow part includes:
a coupling member configured to be capable of being coupled to the medicinal liquid injection valve; and
a medicinal liquid flow line extending from the coupling member to form the guide flow path, and
wherein the medicinal liquid injection apparatus is configured such that when the coupling member is coupled to the medicinal liquid injection valve, the medicinal liquid injection valve is opened by the coupling member to allow the other end of the priming storage space to be connected to the guide flow path.

2. The medicinal liquid injection apparatus of claim 1, wherein the priming storage space is one flow path extending from the one end to the other end.

3. The medicinal liquid injection apparatus of claim 1, wherein the priming storage space is formed to be longer along a movement direction of the medicinal liquid than a width in a direction perpendicular to the movement direction of the medicinal liquid in the priming storage space.

4. The medicinal liquid injection apparatus of claim 1, wherein the priming storage space includes an outer storage space extending downstream at an outside of the medicinal liquid pumping device and including the other end.

5. The medicinal liquid injection apparatus of claim 4, wherein the medicinal liquid storage part further includes a medicinal liquid storage line coupled to the medicinal liquid pumping device to form the outer storage space.

6. The medicinal liquid injection apparatus of claim 1, wherein the priming storage space includes an inner storage space extending upstream at an inside of the medicinal liquid pumping device and including the one end.

7. The medicinal liquid injection apparatus of claim 6, wherein the medicinal liquid pumping device includes:
a chamber configured to accommodate the medicinal liquid therein;
a pressurization unit configured to form the internal space together with the chamber and pressurize the medicinal liquid in the internal space; and
an inner storage part extending from an inner surface of the chamber toward the pressurization unit to form the inner storage space.

8. The medicinal liquid injection apparatus of claim 7, wherein the pressurization unit has a storage corresponding groove formed to engage with the inner storage part.

9. The medicinal liquid injection apparatus of claim 1, wherein the medicinal liquid injection valve includes a valve part configured to be opened in a pressed state of a front end of the valve part and configured to be closed in a released state of the pressing.

10. The medicinal liquid injection apparatus of claim 1, wherein the medicinal liquid flow part further includes a medicinal liquid delivery tube device configured to adjust a flow rate, and
wherein the medicinal liquid injection apparatus further includes an end cap configured to be capable of being coupled to a front end of the medicinal liquid flow part.

11. A medicinal liquid injection preparation method, comprising:
storing a medicinal liquid by introducing the medicinal liquid into an internal space of a medicinal liquid pumping device through a medicinal liquid injection valve;
storing a priming liquid by introducing the priming liquid toward the internal space through the medicinal liquid injection valve;
connecting a flow path by coupling a medicinal liquid flow part to the medicinal liquid injection valve to connect a guide flow path of the medicinal liquid flow part to the internal space; and
priming by pressurizing, by the medicinal liquid pumping device, the internal space to allow the priming liquid flow ahead of the medicinal liquid along the guide flow path.

12. The medicinal liquid injection preparation method of claim 11, wherein in the storing the medicinal liquid, the medicinal liquid is introduced into the internal space through a priming storage space connecting the medicinal liquid injection valve and the internal space, and
wherein in the storing the priming liquid, the priming storage space is filled with the priming liquid.
